(19) United States
(12) Patent Application Publication
(10) Pub. No.: US 2021/0244386 A1
(43) Pub. Date: Aug. 12, 2021

(54) METHODS AND APPARATUSES FOR DETECTION OF ONE OR MORE TAPS BY AN ULTRASOUND DEVICE

(71) Applicants: Kirthi Bellamkonda, New Haven, CT (US); Krishna Ersson, Guilford, CT (US); Matthew de Jonge, Brooklyn, NY (US); Amy Wilkinson, San Francisco, CA (US); Landry Collet, Vandoeuvre-lès-Nancy (FR); John E. Peabody, JR., Hamden, CT (US)

(72) Inventors: Kirthi Bellamkonda, New Haven, CT (US); Krishna Ersson, Guilford, CT (US); Matthew de Jonge, Brooklyn, NY (US); Amy Wilkinson, San Francisco, CA (US); Landry Collet, Vandoeuvre-lès-Nancy (FR); John E. Peabody, JR., Hamden, CT (US)

(73) Assignee: Butterfly Network, Inc., Guilford, CT (US)

(21) Appl. No.: 17/168,122

(22) Filed: Feb. 4, 2021

Related U.S. Application Data
(60) Provisional application No. 62/971,546, filed on Feb. 7, 2020.

(51) Int. Cl. A61B 8/08; A61B 8/00

(52) U.S. Cl. CPC A61B 8/5207; A61B 2562/0219; A61B 8/56; A61B 8/54

(57) ABSTRACT

Aspects of the technology described herein relate to detection of one or more taps by an ultrasound device. An ultrasound device may comprise tap detection circuitry and be configured to detect, with the tap detection circuitry, one or more taps on an exterior of the ultrasound device during ultrasound imaging, and transmit an indication of the detection of the one or more taps to a processing device in operative communication with the ultrasound device during the ultrasound imaging. A processing device in operative communication with the ultrasound device may be configured to receive, from the ultrasound device, an indication of detection of one or more taps on an exterior of the ultrasound device during ultrasound imaging, and perform an action controlling an aspect of the ultrasound imaging based on receiving the indication of the detection of the one or more taps from the ultrasound device.

### CROSS-REFERENCE TO RELATED APPLICATIONS

The present application claims the benefit under 35 U.S.C. § 119(e) of U.S. patent application Ser. No. 62/971,546, filed Feb. 7, 2020 under Attorney Docket No. B1348.70173US00, and entitled “METHODS AND APPARATUSES FOR DETECTION OF ONE OR MORE TAPS BY AN ULTRASOUND DEVICE,” which is hereby incorporated by reference herein in its entirety.

### FIELD

Generally, the aspects of the technology described herein relate to ultrasound devices. Some aspects relate to detection of one or more taps on the ultrasound device by the ultrasound device itself.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the present application, an ultrasound device comprises tap detection circuitry and is configured to: detect, with the tap detection circuitry, one or more taps on an exterior of the ultrasound device during ultrasound imaging; and transmit an indication of the detection of the one or more taps to a processing device in operative communication with the ultrasound device during the ultrasound imaging.

In some embodiments, the tap detection circuitry includes a chip comprising an accelerometer and circuitry configured to internally process acceleration data from the accelerometer to determine that the acceleration data represents the one or more taps, and output an indication of detection of the one or more taps as one or more bits. In some embodiments, the tap detection circuitry is configured to differentiate between single taps and double taps.

In some embodiments, the ultrasound device is configured, when detecting the one or more taps during the ultrasound imaging and transmitting the indication of the detection of the one or more taps during the ultrasound imaging, to detect the one or more taps and transmit the indication of the detection of the one or more taps after a session of the ultrasound imaging has begun. In some embodiments, the ultrasound device is configured, when detecting the one or more taps during the ultrasound imaging and transmitting the indication of the detection of the one or more taps during the ultrasound imaging, to detect the one or more taps and transmit the indication of the detection of the one or more taps after the ultrasound device has begun to collect ultrasound data. In some embodiments, the ultrasound device is configured, when detecting the one or more taps during the ultrasound imaging and transmitting the indication of the detection of the one or more taps during the ultrasound imaging, to detect the one or more taps and transmit the indication of the detection of the one or more taps while the ultrasound device is placed on a subject for the ultrasound imaging.

In some embodiments, the tap detection circuitry is configured to detect the one or more taps using a threshold acceleration between or equal approximately 0-62.5 mg. In some embodiments, the tap detection circuitry is configured to detect the one or more taps using a threshold acceleration between or equal approximately 62.5-125 mg. In some embodiments, the tap detection circuitry is configured to detect the one or more taps using a threshold acceleration between or equal approximately 125-187.5 mg.

According to another aspect of the present application, a processing device in operative communication with an ultrasound device is configured to: receive, from the ultrasound device, an indication of detection of one or more taps on an exterior of the ultrasound device during ultrasound imaging; and perform an action controlling an aspect of the ultrasound imaging based on receiving the indication of the detection of the one or more taps from the ultrasound device.

In some embodiments, the processing device is configured, when performing the action controlling the aspect of the ultrasound imaging, to: control recording of a cine, freeze a current ultrasound image on a screen of the processing device, save to memory an ultrasound image that is frozen on the screen of the processing device, modify an imaging depth, modify a gain, toggle color Doppler imaging on or off, switch imaging modes, and/or switch imaging presets.

In some embodiments, the processing device is configured, when performing the action, to modify the processing device's own configuration. In some embodiments, the processing device is configured, when performing the action, to modify the ultrasound device's configuration. In some embodiments, the processing device is configured, when performing the action, to modify both the processing device's own configuration and the ultrasound device's configuration.

In some embodiments, the processing device is further configured to provide a user with options for the action that the processing device will be configured to perform based on receiving the indication of the detection of the one or more taps on the exterior of the ultrasound device. In some embodiments, the processing device is further configured to provide a user with options for a threshold tap that the ultrasound device will use to detect one or more taps. In some embodiments, the processing device is further configured to provide a user with options for timing that the ultrasound device will use to detect double taps. In some embodiments, the processing device is further configured to provide a user with options for a duration that the processing device will be configured to perform the action based on receiving the indication of the detection of the one or more taps on the exterior of the ultrasound device.

In some embodiments, the processing device is configured, when receiving the indication of the detection of the one or more taps during the ultrasound imaging, to receive the indication of the detection of the one or more taps after a session of the ultrasound imaging has begun. In some embodiments, the processing device is configured, when receiving the indication of the detection of the one or more taps during the ultrasound imaging, to receive the indication of the detection of the one or more taps after the ultrasound device has begun to collect ultrasound data. In some embodiments, the processing device is configured, when receiving the indication of the detection of the one or more taps during the ultrasound imaging, to receive the indication of the detection of the one or more taps while the ultrasound device is placed on a subject for the ultrasound imaging. In some embodiments, the action does not comprise initiating the ultrasound imaging.

Some aspects include a method of performing the above functions of the ultrasound device or processing device. Some aspects include at least one non-transitory computer-readable storage medium storing processor-executable instructions that, when executed by at least one processor on an ultrasound device, cause the at least one processor to perform the above functions of the processing device.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a schematic block diagram of an example ultrasound system, in accordance with certain embodiments described herein;

FIG. 2 illustrates three axes of a non-limiting example of an ultrasound device, in accordance with certain embodiments described herein;

FIG. 3 illustrates a process for detecting one or more taps on an ultrasound device, in accordance with certain embodiments described herein; and

FIG. 4 illustrates a process for performing one or more actions based on detecting one or more taps on an ultrasound device, in accordance with certain embodiments described herein.

### DETAILED DESCRIPTION

Certain embodiments of ultrasound systems include an ultrasound device (e.g., a handheld ultrasound probe) and a processing device in operative communication with the ultrasound device (e.g., across a wired or wireless communication link). The processing device (e.g., a phone or tablet) may display a graphical user interface (GUI) through which a user may view recently collected ultrasound images and control aspects of ultrasound imaging. For example, a user may control recording of a cine (i.e., a sequence of ultrasound images), freezing the current ultrasound image on the screen of the processing device, saving to memory the ultrasound image that is frozen on the screen, modifying the imaging depth, modifying the gain, toggling color Doppler imaging on or off, switching imaging modes (e.g., biplane, spectra Doppler, or power Doppler imaging modes), and switching imaging presets (e.g., sets of imaging parameters optimized for imaging particular anatomies).

In some embodiments, it may be helpful to enable a user to control such aspects of ultrasound imaging without needing to interact with a GUI displayed on the display screen of the processing device. For example, touching the display screen of the processing device may interfere with the user's sterility. As another example, a user may have two hands occupied (e.g., one hand holding the ultrasound device and another hand holding an instrument such as a needle) and thus may not have a free hand to touch the display screen. As another example, a user may be wearing gloves on his/her hands, and the processing device may not register touches to the display screen by gloved hands. In some cases, removing gloves in order to interact with a GUI may not be desirable, because it may be desirable for the user to execute the current clinical procedure as swiftly as possible. Particularly in anesthesia and interventional radiology, reduced procedure time may be associated with reduced infection and complication risk as well as increased throughput.

The inventors have recognized that including tap detection circuitry in an ultrasound device (e.g., a handheld ultrasound probe) may enable the ultrasound device to detect one or more taps on the exterior of the ultrasound device. The tap circuitry in the ultrasound device may be configured to detect one or more taps on the exterior of the ultrasound device during ultrasound imaging without an adverse impact on the ultrasound imaging. In other words, contrary to conventional wisdom that discourages bumping or otherwise vibrating an ultrasound device due to concern of the negative consequences on the imaging, aspects of the present application provide an ultrasound device with tap detection circuitry, wherein the tap circuitry may be configured to detect one or more taps after an ultrasound imaging session has begun, after the ultrasound device has begun to collect ultrasound data, and/or while the ultrasound device is placed on the subject for ultrasound imaging. The tap circuitry may be configured to detect taps with sufficient sensitivity such that the taps need not be so forceful so as to interfere with the ultrasound device's imaging. In particular, the tap detection circuitry may be configured to detect taps using a sufficiently low threshold acceleration that the imaging is not adversely impacted.

In response to detecting one or more taps on the exterior of the ultrasound device, the ultrasound device may be configured to transmit an indication of this detection to the processing device during the ultrasound imaging. In response to receiving this indication, the processing device may be configured to perform an action related to controlling an aspect of ultrasound imaging, during the imaging itself. When the tap circuitry is configured to detect taps with sufficient sensitivity such that the taps need not be so forceful so as to interfere with the ultrasound device's imaging, the action performed by the processing device in response to the taps may be actions controlling aspects of the ultrasound imaging, and not just initiation of ultrasound imaging. For example, the action may be to initiate or stop recording of a cine, freeze the current ultrasound image on the screen of the processing device, save to memory the ultrasound image that is frozen on the screen, modify the imaging depth, modify the gain, toggle color Doppler imaging on or off, switch imaging modes, or switch imaging presets. In some embodiments, in performing the action, the processing device may modify its own configuration. In some embodiments, in performing the action, the processing device may modify configuration of the ultrasound device (e.g., by transmitting a command to the ultrasound device). In some embodiments, in performing the action, the processing device may modify configuration both of the processing device itself and the ultrasound device. Thus, a user may be able to control certain aspects of ultrasound imaging by tapping on the ultrasound device, and without needing to touch the display screen of the processing device. Using taps on the exterior of the ultrasound device, rather than pressing a button on the exterior of the ultrasound device, to control aspects of ultrasound imaging may be helpful because buttons may represent possible access points for leakage of liquid into the ultrasound device.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

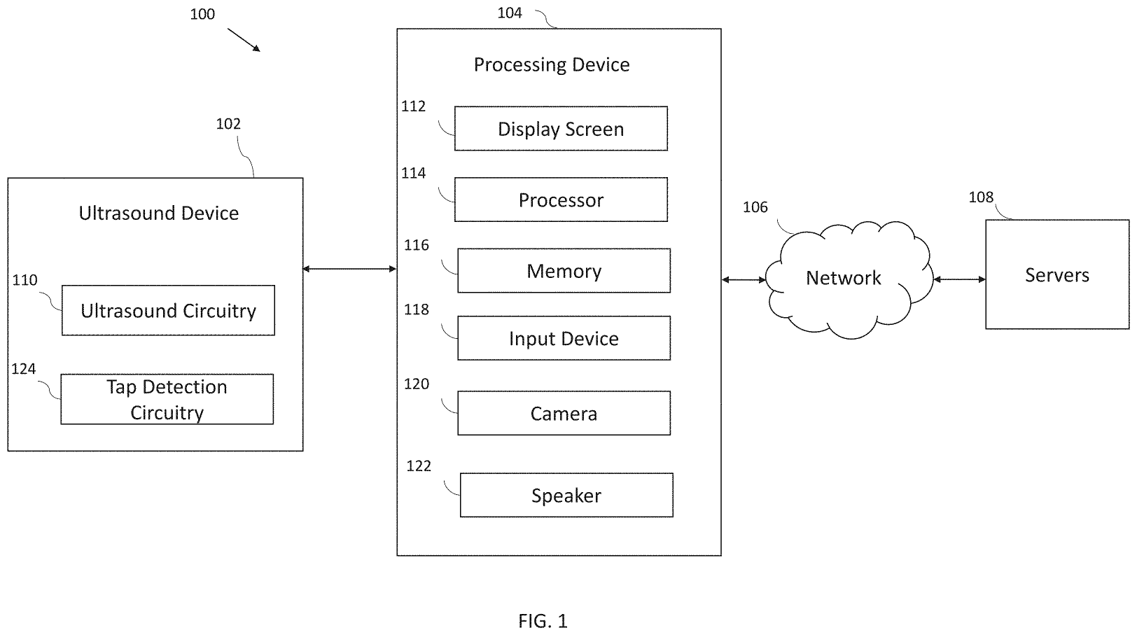

FIG. 1 illustrates a schematic block diagram of an example ultrasound system 100, in accordance with certain embodiments described herein. The ultrasound system 100 includes an ultrasound device 102 and a processing device 104.

The ultrasound device 102 includes ultrasound circuitry 110 and tap detection circuitry 124. The processing device 104 includes a display screen 112, a processor 114, a memory 116, an input device 118, a camera 120, and a speaker 122. The processing device 104 is in wired (e.g., through a lightning connector or a mini-USB connector) and/or wireless communication (e.g., using BLUETOOTH, ZIGBEE, and/or WiFi wireless protocols) with the ultrasound device 102.

The ultrasound device 102 may be configured to generate ultrasound data that may be employed to generate an ultrasound image. The ultrasound device 102 may be constructed in any of a variety of ways. In some embodiments, the ultrasound device 102 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 110 may be configured to generate the ultrasound data. The ultrasound circuitry 110 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 110 (e.g., transmit circuitry, receive circuitry, control circuitry, power management circuitry, and processing circuitry) to form a monolithic ultrasound device. The ultrasound device 102 may transmit ultrasound data and/or ultrasound images to the processing device 104 over a wired (e.g., through a lightning connector or a mini-USB connector) and/or wireless (e.g., using BLUETOOTH, ZIGBEE, and/or WiFi wireless protocols) communication link.

The tap detection circuitry 124 may be configured to detect one or more taps on the exterior of the ultrasound device 102. In some embodiments, the tap detection circuitry 124 may include an accelerometer and processing circuitry to process acceleration data from the accelerometer and detect one or more taps based on the acceleration data. In some embodiments, the tap detection circuitry 124 may be configured to detect single taps. To detect single taps, the tap detection circuitry 124 may be configured to determine if the acceleration as measured by the accelerometer exceeds a threshold acceleration and falls below the threshold acceleration within a time window. In some embodiments, the tap detection circuitry 124 may be configured to detect double taps. To detect double taps, the tap detection circuitry 124 may be configured to detect if there are two consecutive single taps where the time between the two single taps is longer than a quiet time window and is shorter than a latency time window. The tap detection circuitry 124 may detect the single taps in the manner described above. In some embodiments, the tap detection circuitry 124 may be configured to detect and differentiate between single and double taps.

The tap circuitry 124 may be configured to detect one or more taps on the exterior of the ultrasound device 102 during ultrasound imaging. In other words, the tap circuitry may be configured to detect one or more taps after an ultrasound imaging session has begun, after the ultrasound device 102 has begun to collect ultrasound data, and/or while the ultrasound device 102 is placed on the subject for ultrasound imaging. The tap circuitry 124 may be configured to be detect taps with sufficient sensitivity such that the taps need not be so forceful so as to interfere with the ultrasound device 102's imaging. In particular, the tap detection circuitry 124 may be configured to detect taps using a sufficiently low threshold acceleration. In some embodiments, the threshold acceleration may be between or equal approximately 0-62.5 mg (where g is the acceleration due to gravity). In some embodiments, the threshold acceleration may be between or equal to approximately 62.5-125 mg. In some embodiments, the threshold acceleration may be between or equal to approximately 125-187.5 mg. For example, the threshold acceleration may be 62.5 mg, 125 mg, or 187.5 mg.

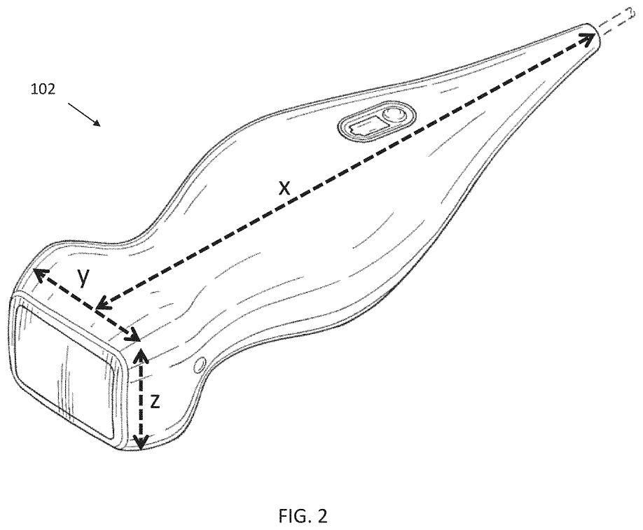

In embodiments in which the tap detection circuitry 124 includes an accelerometer, the accelerometer may be a three-axis accelerometer, and the tap detection circuitry 124 may be configured to detect taps along any of the three axes based on acceleration data along any of the three axes. FIG. 2 illustrates three axes (x, y, and z) of the ultrasound device 102, in accordance with certain embodiments described herein. In some embodiments, the tap detection circuitry 124 may not be configured to detect taps along the x-axis (e.g., along the longitudinal axis of the ultrasound device 102). This may be because during imaging, tapping along the x-axis may interfere with the imaging.

In some embodiments, the tap detection circuitry 124 may be a chip such as the LIS2DW12 (produced by STMicroelectronics). Such a chip may include an accelerometer and processing circuitry configured to internally process acceleration data from the accelerometer to determine whether the acceleration data represents one or more taps (e.g., using the manner described above), and output an indication of detection of one or more taps as one or more bits. In some embodiments, the tap detection circuitry 124 may include an accelerometer and processing circuitry external to the accelerometer (e.g., in a different chip than the accelerometer) configured to determine whether acceleration data represents taps (e.g., using the manner described above) and to output an indication of detection of one or more taps as one or more bits. In some embodiments, the tap detection circuitry 124 may include an inertial motion unit (IMU). The IMU may include an accelerometer, a gyroscope, and a magnetometer, each having one, two, or three axes. The tap detection circuitry 124 may further include processing circuitry external to the accelerometer (e.g., in a different chip than the IMU) configured to determine whether acceleration data represents taps (e.g., using the manner described above) and to output an indication of detection of one or more taps as one or more bits. An IMU may also be capable of detecting motion representing events besides taps, such as gestures (e.g., translation or rotation) performed with the ultrasound device 102. Processing circuitry in the ultrasound device 102 may be configured to process motion detected by the IMU in order to determine that the motion represents a gesture, and then transmit an indication of the gesture to the processing device 104.

In some embodiments, the tap detection circuitry 124 may be disposed within the ultrasound device 102 at a location where a user tends to hold the ultrasound device 102. In some embodiments, the tap detection circuitry 124 may be disposed within the ultrasound device 102 at a location that is far from the axis of the ultrasound device 102 that rotates in response to taps. For example, such a location may be at the tail of the ultrasound device 102.

Referring back to FIG. 1, in some embodiments, upon detecting a single or double tap by the tap detection circuitry 124 during ultrasound imaging, the ultrasound device 102 may be configured to transmit an indication of the single or double tap (e.g., an interrupt signal) to the processing device 104 during the ultrasound imaging. In some embodiments, the tap detection circuitry 124 may be powered such that it may detect taps even at a time when the ultrasound device 102 is not performing imaging. In some embodiments, rather than processing circuitry in the tap detection circuitry 124 processing acceleration or IMU data to detect taps or gestures and transmitting indications of detection of taps or gestures to the processing device 104, the ultrasound device 102 may be configured to transmit acceleration or IMU data to the processing device 104 during ultrasound imaging, and the processing device 104 may be configured to process the acceleration or IMU data to detect taps or gestures. In such embodiments, the ultrasound device 102 may include an accelerometer or IMU without processing circuitry to process the acceleration or IMU data to detect taps or gestures.

Referring now to the processing device 104, the processor 114 may include specially-programmed and/or special-purpose hardware such as an application-specific integrated circuit (ASIC). For example, the processor 114 may include one or more graphics processing units (GPUs) and/or one or more tensor processing units (TPUs). TPUs may be ASICs specifically designed for machine learning (e.g., deep learning). The TPUs may be employed, for example, to accelerate the inference phase of a neural network. The processing device 104 may be configured to process the ultrasound data received from the ultrasound device 102 to generate ultrasound images for display on the display screen 112. The processing may be performed by, for example, the processor 114. The processor 114 may also be adapted to control the acquisition of ultrasound data with the ultrasound device 102. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 104 may be configured to perform certain of the processes (e.g., the process 400) described herein using the processor 114 (e.g., one or more computer hardware processors) and one or more articles of manufacture that include non-transitory computer-readable storage media such as the memory 116. The processor 114 may control writing data to and reading data from the memory 116 in any suitable manner. To perform certain of the processes described herein, the processor 114 may execute one or more processor-executable instructions stored in one or more non-transitory computer-readable storage media (e.g., the memory 116), which may serve as non-transitory computer-readable storage media storing processor-executable instructions for execution by the processor 114. The camera 120 may be configured to detect light (e.g., visible light) to form an image. The camera 120 may be on the same face of the processing device 104 as the display screen 112. The display screen 112 may be configured to display images and/or videos, and may be, for example, a liquid crystal display (LCD), a plasma display, and/or an organic light emitting diode (OLED) display on the processing device 104. The input device 118 may include one or more devices capable of receiving input from a user and transmitting the input to the processor 114. For example, the input device 118 may include a keyboard, a mouse, a microphone, touch-enabled sensors on the display screen 112, and/or a microphone. The display screen 112, the input device 118, the camera 120, and the speaker 122 may be communicatively coupled to the processor 114 and/or under the control of the processor 114.

It should be appreciated that the processing device 104 may be implemented in any of a variety of ways. For example, the processing device 104 may be implemented as a handheld device such as a mobile smartphone or a tablet. Thereby, a user of the ultrasound device 102 may be able to operate the ultrasound device 102 with one hand and hold the processing device 104 with another hand. In other examples, the processing device 104 may be implemented as a portable device that is not a handheld device, such as a laptop. In yet other examples, the processing device 104 may be implemented as a stationary device such as a desktop computer. The processing device 104 may be connected to the network 106 over a wired connection (e.g., via an Ethernet cable) and/or a wireless connection (e.g., over a WiFi network). The processing device 104 may thereby communicate with (e.g., transmit data to or receive data from) the one or more servers 108 over the network 106. For example, a party may provide from the server 108 to the processing device 104 processor-executable instructions for storing in one or more non-transitory computer-readable storage media which, when executed, may cause the processing device 104 to perform certain of the processes (e.g., the process 400) described herein. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the instant application).

FIG. 1 should be understood to be non-limiting. For example, the ultrasound system 100 may include fewer or more components than shown and the processing device 104 and ultrasound device 102 may include fewer or more components than shown. In some embodiments, the processing device 104 may be part of the ultrasound device 102.

In some embodiments, the processing device 104 may be configured to perform one or more actions based on receiving, from the ultrasound device 102, an indication of the detection of one or more taps by the tap detection circuitry 124 during ultrasound imaging (after an ultrasound imaging session has begun, after the ultrasound device 102 has begun to collect ultrasound data, and/or while the ultrasound device 102 is placed on the subject for ultrasound imaging). When the tap circuitry 124 is configured to detect taps with sufficient sensitivity such that the taps need not be so forceful so as to interfere with the ultrasound device's imaging (as described above), the action performed by the processing device 104 in response to the taps may be actions controlling aspects of the ultrasound imaging, and not just initiation of ultrasound imaging. In some embodiments, the action may be either to initiate recording of a cine (i.e., a sequence of ultrasound images) or to stop recording of a cine that was previously initiated. In some embodiments, the action may be either to freeze the current ultrasound image on the screen of the processing device or to save to memory the ultrasound image that is frozen on the screen. In some embodiments, the action may be to modify the imaging depth. For example, in some embodiments, the action may be to switch between a shallow imaging depth and a deep imaging depth, where the shallow and deep imaging depths may be predetermined for a given imaging preset (i.e., a set of imaging parameters optimized for imaging particular anatomy). In some embodiments, the action may be to modify the gain. For example, in some embodiments, there may be a predetermined sequence of gains, and the action may be to advance one gain in the sequence. In some embodiments, the action may be to toggle color Doppler imaging on or off. In some embodiments, the action may be to switch imaging modes (e.g., biplane, spectra Doppler, or power Doppler imaging modes). For example, in some embodiments, there may be a predetermined sequence of imaging modes, and the action may be to advance one imaging mode in the sequence. In some embodiments, the action may be to switch between imaging presets. The action may include the processing device 104 modifying its own configuration, modifying the configuration of the ultrasound device 102, or both.

In some embodiments, the processing device 104 may be configured to provide a user with options for actions that the processing device 104 may be configured to perform based on receiving the indication of the detection of the one or more taps from the ultrasound device 102. In some embodiments, the processing device 104 may be configured to provide these options upon detecting connection of the ultrasound device 102 to the processing device 104 for the first time. In response to receiving a user selection of an action option, the processing device 104 may be configured to configure itself to perform the action based on receiving the indication of the detection of the one or more taps from the ultrasound device 102. In some embodiments, if the user does not select an action option, the processing device 104 may be configured to select a default action option (e.g., capturing a cine).

In some embodiments, the processing device 104 may be configured to provide a user with options for the threshold tap strength (e.g., the threshold acceleration described above) that the ultrasound device 102 may use to detect taps. In some embodiments, the processing device 104 may be configured to provide these options upon detecting connection of the ultrasound device 102 to the processing device 104 for the first time. In some embodiments, if the user does not select a tap strength option, the processing device 104 may be configured to select a default tap strength option (e.g., a medium strength).

In some embodiments, the processing device 104 may be configured to provide a user with options for timing (e.g., the length of the quiet time window and the length of the latency time window) that the ultrasound device 102 may use to detect double taps. In some embodiments, the processing device 104 may be configured to provide these options upon detecting connection of the ultrasound device 102 to the processing device 104 for the first time. In some embodiments, if the user does not select a timing option, the processing device 104 may be configured to select a default timing option (e.g., a certain length of time for the quite time window and the latency time window).

In some embodiments, the processing device 104 may be configured to provide a user with options for the duration that detection of one or more taps causes an action to be performed. For example, the duration may include a certain number of minutes (e.g., 5, 15, or 30 minutes) after the user turns on the tap functionality, the length of the study performed after the user turns on the tap functionality, or until the user turns off the tap functionality. In some embodiments, the processing device 104 may be configured to provide these options upon detecting connection of the ultrasound device 102 to the processing device 104 for the first time. In some embodiments, if the user does not select a duration option, the processing device 104 may be configured to select a default duration option (e.g., a certain number of minutes, such as 15).

While the above description has described actions that the processing device 104 may be configured to perform based on detection of taps by the ultrasound device 102, in embodiments in which the ultrasound device 102 includes an IMU, the processing device 104 may be configured to perform such actions based on detection by the IMU and the ultrasound device 102. of a gesture (e.g., translation or rotation) performed with the ultrasound device 102.

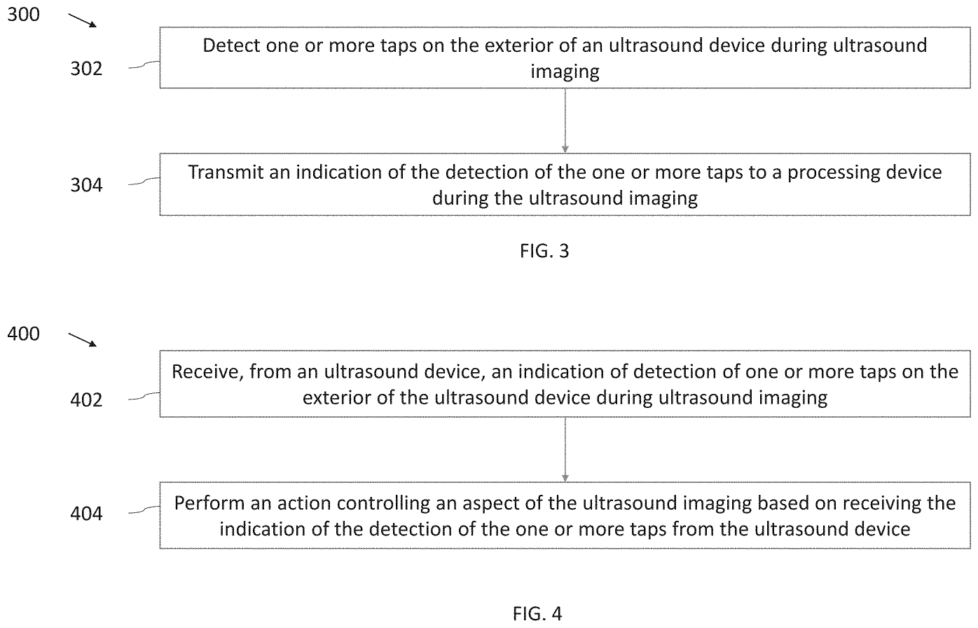

FIG. 3 illustrates a process 300 for detecting one or more taps on an ultrasound device (e.g., the ultrasound device 102) during ultrasound imaging, in accordance with certain embodiments described herein. The process 300 may be performed by the ultrasound device.

In act 302, the ultrasound device detects one or more taps on the exterior of the ultrasound device during ultrasound imaging (e.g., after the ultrasound device has begun to collect ultrasound data, and/or while the ultrasound device is placed on the subject for ultrasound imaging). In some embodiments, the ultrasound device may be configured to detect single taps. In some embodiments, the ultrasound device may be configured to detect double taps. In some embodiments, the ultrasound device may be configured to detect and differentiate between single and double taps. The ultrasound device may use tap detection circuitry (e.g., the tap detection circuitry 124) to detect the taps. The tap circuitry may be configured to be detect taps with sufficient sensitivity such that the taps need not be so forceful so as to interfere with the ultrasound device imaging. In particular, the tap detection circuitry may be configured to detect taps using a sufficiently low threshold acceleration. In some embodiments, the threshold acceleration may be between or equal approximately 0-62.5 mg (where g is the acceleration due to gravity). In some embodiments, the threshold acceleration may be between or equal to approximately 62.5-125 mg. In some embodiments, the threshold acceleration may be between or equal to approximately 125-187.5 mg. For example, the threshold acceleration may be 62.5 mg, 125 mg, or 187.5 mg. Further description of detecting taps may be found with reference to the tap detection circuitry 124. The process 300 proceeds from act 302 to act 304.

In act 304, the ultrasound device transmits an indication of the detection of the one or more taps to a processing device (e.g., the processing device 104) during the ultrasound imaging. The processing device may be in operative communication with the ultrasound device. Transmitting the indication may include transmitting an interrupt signal during the ultrasound imaging. In some embodiments, the transmitted indication may include an indication of whether a single or double tap was detected. In response to receiving the indication of the one or more taps, the processing device may perform one or actions, as described with reference to the process 400.

In some embodiments, the ultrasound device may not transmit an indication of the detection of the one or more taps after a certain duration. For example, the duration may include a certain number of minutes (e.g., 5, 15, or 30 minutes) after the user turns on the tap functionality, the length of the study performed after the user turns on the tap functionality, or until the user turns off the tap functionality.

While the above description has described the ultrasound device transmitting an indication of detection of one or more taps to the processing device, in embodiments in which the ultrasound device includes an IMU, the ultrasound device may transmit an indication of detection of a gesture (e.g., translation or rotation) performed with the ultrasound device to the processing device.

FIG. 4 illustrates a process 400 for performing one or more actions controlling aspects of ultrasound imaging based on detecting one or more taps on an ultrasound device (e.g., the ultrasound device 102) during the ultrasound imaging, in accordance with certain embodiments described herein. The process 400 may be performed by a processing device (e.g., the processing device 104) in operative communication with the ultrasound device.

In act 402, the processing device receives from the ultrasound device an indication of detection of one or more taps on the exterior of the ultrasound device during ultrasound imaging (e.g., after the ultrasound device has begun to collect ultrasound data, and/or while the ultrasound device is placed on the subject for ultrasound imaging). Further description of the ultrasound device detecting one or more taps and transmitting the indication of the detection of the one or more taps may be found with reference to the process 300. The process 400 proceeds from act 402 to act 404.

In act 404, the processing device performs one or more actions based on receiving the indication of the detection of the one or more taps from the ultrasound device. The action may be an action controlling an aspect of the ultrasound imaging (and not just initiation of ultrasound imaging). In some embodiments, the action may be either to initiate recording of a cine (i.e., a sequence of ultrasound images) or to stop recording of a cine that was previously initiated. In some embodiments, the action may be either to freeze the current ultrasound image on the screen of the processing device or to save to memory the ultrasound image that is frozen on the screen. In some embodiments, the action may be to modify the imaging depth. For example, in some embodiments, the action may be to switch between a shallow imaging depth and a deep imaging depth, where the shallow and deep imaging depths may be predetermined for a given imaging preset (i.e., a set of imaging parameters optimized for imaging particular anatomy). In some embodiments, the action may be to modify the gain. For example, in some embodiments, there may be a predetermined sequence of gains, and the action may be to advance one gain in the sequence. In some embodiments, the action may be to toggle color Doppler imaging on or off. In some embodiments, the action may be to switch imaging modes (e.g., biplane, spectra Doppler, or power Doppler imaging modes). For example, in some embodiments, there may be a predetermined sequence of imaging modes, and the action may be to advance one imaging mode in the sequence. In some embodiments, the action may be to switch between presets (e.g., sets of imaging parameters optimized for imaging particular anatomy). The action may include the processing device modifying its own configuration of the processing device, modifying configuration of the ultrasound device, or both.

In some embodiments, the processing device may not perform actions based on receiving the indication of the detection of the one or more taps from the ultrasound device after a certain duration. For example, the duration may include a certain number of minutes (e.g., 5, 15, or 30 minutes) after the user turns on the tap functionality, the length of the study performed after the user turns on the tap functionality, or until the user turns off the tap functionality.

In some embodiments, rather than the processing device receiving from the ultrasound device, at act 402, an indication of detection of one or more taps on the exterior of the ultrasound device, the processing device may receive acceleration or IMU data from the ultrasound device. The processing device may then process the acceleration or IMU data to determine that the data represents one or more taps. At act 404, the processing device may then perform one or more actions based on determining, from the acceleration or IMU data, that the data represents one or more taps.

While the above description has described actions that the processing device may perform based on receiving an indication of detection of taps from the ultrasound device, in embodiments in which the ultrasound device includes an IMU, the processing device may perform such actions based on receiving from the ultrasound device an indication of detection of a gesture (e.g., translation or rotation) performed with the ultrasound device.

According to an aspect of the present application, a method is provided, comprising: detecting, with the tap detection circuitry in an ultrasound device, one or more taps on an exterior of the ultrasound device during ultrasound imaging; and transmitting, from the ultrasound device, an indication of the detection of the one or more taps to a processing device in operative communication with the ultrasound device during the ultrasound imaging.

In some embodiments, the tap detection circuitry includes a chip comprising an accelerometer and circuitry configured to internally process acceleration data from the accelerometer to determine that the acceleration data represents the one or more taps, and output an indication of detection of the one or more taps as one or more bits.

In some embodiments, detecting the one or more taps on the exterior of the ultrasound device during the ultrasound imaging includes differentiating between single taps and double taps.

In some embodiments, detecting the one or more taps during the ultrasound imaging and transmitting the indication of the detection of the one or more taps during the ultrasound imaging includes detecting the one or more taps and transmitting the indication of the detection of the one or more taps after a session of the ultrasound imaging has begun. In some embodiments, detecting the one or more taps during the ultrasound imaging and transmitting the indication of the detection of the one or more taps during the ultrasound imaging includes detecting the one or more taps and transmitting the indication of the detection of the one or more taps after the ultrasound device has begun to collect ultrasound data. In some embodiments, detecting the one or more taps during the ultrasound imaging and transmitting the indication of the detection of the one or more taps during the ultrasound imaging includes detecting the one or more taps and transmitting the indication of the detection of the one or more taps while the ultrasound device is placed on a subject for the ultrasound imaging.

In some embodiments, detecting the one or more taps during the ultrasound imaging includes using a threshold acceleration. For example, the threshold acceleration may be between or equal approximately 0-62.5 mg, between or equal approximately 62.5-125 mg, and/or between or equal approximately 125-187.5 mg.

According to an aspect of the present application, a method is provided, comprising: receiving, by a processing device in operative communication with an ultrasound device and from the ultrasound device, an indication of detection of one or more taps on an exterior of the ultrasound device during ultrasound imaging; and performing, by the processing device an action controlling an aspect of the ultrasound imaging based on receiving the indication of the detection of the one or more taps from the ultrasound device.

In some embodiments, performing the action controlling the aspect of the ultrasound imaging may include controlling recording of a cine, freezing a current ultrasound image on a screen of the processing device, saving to memory an ultrasound image that is frozen on the screen of the processing device, modifying an imaging depth, modifying a gain, toggling color Doppler imaging on or off, switching imaging modes, and/or switching imaging presets.

According to some embodiments, performing the action includes modifying the processing device's own configuration. In some embodiments, performing the action includes modifying the ultrasound device's configuration. In some embodiments, performing the action includes modifying both the processing device's own configuration and the ultrasound device's configuration.

According to some embodiments, the method may further include providing a user with options for the action that the processing device will be configured to perform based on receiving the indication of the detection of the one or more taps on the exterior of the ultrasound device. In some embodiments, the method may further include providing a user with options for a threshold tap that the ultrasound device will use to detect one or more taps. In some embodiments, the method may further include providing a user with options for timing that the ultrasound device will use to detect double taps. In some embodiments, the method further includes providing a user with options for a duration that the processing device will be configured to perform the action based on receiving the indication of the detection of the one or more taps on the exterior of the ultrasound device.

In some embodiments, receiving the indication of the detection of the one or more taps during the ultrasound imaging includes receiving the indication of the detection of the one or more taps after a session of the ultrasound imaging has begun. In some examples, receiving the indication of the detection of the one or more taps during the ultrasound imaging includes receiving the indication of the detection of the one or more taps after the ultrasound device has begun to collect ultrasound data.

According to some embodiments, receiving the indication of the detection of the one or more taps during the ultrasound imaging includes receiving the indication of the detection of the one or more taps while the ultrasound device is placed on a subject for the ultrasound imaging.

According to some embodiments, the action does not comprise initiating the ultrasound imaging.

According to an aspect of the present application, at least one non-transitory computer-readable storage medium storing processor-executable instructions that, when executed by at least one processor on a processing device in operative communication with an ultrasound device, cause the at least one processor to: receive, from the ultrasound device, an indication of detection of one or more taps on an exterior of the ultrasound device during ultrasound imaging; and perform an action controlling an aspect of the ultrasound imaging based on receiving the indication of the detection of the one or more taps from the ultrasound device.

In some embodiments, instructions for performing the action controlling the aspect of the ultrasound imaging include instructions for controlling recording of a cine, freezing a current ultrasound image on a screen of the processing device, saving to memory an ultrasound image that is frozen on the screen of the processing device, modifying an imaging depth, modifying a gain, toggling color Doppler imaging on or off, switching imaging modes, and/or switching imaging presets. In some embodiments, the instructions for performing the action include instructions for modifying the processing device's own configuration and/or instructions for modifying the ultrasound device's configuration.

In some embodiments, the at least one non-transitory computer-readable storage medium may further store instructions for providing a user with options for the action that the processing device will be configured to perform based on receiving the indication of the detection of the one or more taps on the exterior of the ultrasound device. In some embodiments, the at least one non-transitory computer-readable storage medium may further store instructions for providing a user with options for a threshold tap that the ultrasound device will use to detect one or more taps. In some embodiments, the at least one non-transitory computer-readable storage medium may further store instructions for providing a user with options for timing that the ultrasound device will use to detect double taps. In some embodiments, the at least one non-transitory computer-readable storage medium may further store instructions for providing a user with options for a duration that the processing device will be configured to perform the action based on receiving the indication of the detection of the one or more taps on the exterior of the ultrasound device.

In some examples, the action does not comprise initiating the ultrasound imaging.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound device, comprising:
tap detection circuitry;
and configured to:
detect, with the tap detection circuitry, one or more taps on an exterior of the ultrasound device during ultrasound imaging; and
transmit an indication of the detection of the one or more taps to a processing device in operative communication with the ultrasound device during the ultrasound imaging.

2. The ultrasound device of claim 1, wherein the tap detection circuitry includes a chip comprising an accelerometer and circuitry configured to internally process acceleration data from the accelerometer to determine that the acceleration data represents the one or more taps, and output an indication of detection of the one or more taps as one or more bits.

3. The ultrasound device of claim 1, wherein the tap detection circuitry is configured to differentiate between single taps and double taps.

4. The ultrasound device of claim 1, wherein the ultrasound device is configured, when detecting the one or more taps during the ultrasound imaging and transmitting the indication of the detection of the one or more taps during the ultrasound imaging, to detect the one or more taps and transmit the indication of the detection of the one or more taps after a session of the ultrasound imaging has begun.

5. The ultrasound device of claim 1, wherein the ultrasound device is configured, when detecting the one or more taps during the ultrasound imaging and transmitting the indication of the detection of the one or more taps during the ultrasound imaging, to detect the one or more taps and transmit the indication of the detection of the one or more taps after the ultrasound device has begun to collect ultrasound data.

6. The ultrasound device of claim 1, wherein the ultrasound device is configured, when detecting the one or more taps during the ultrasound imaging and transmitting the indication of the detection of the one or more taps during the ultrasound imaging, to detect the one or more taps and transmit the indication of the detection of the one or more taps while the ultrasound device is placed on a subject for the ultrasound imaging.

7. The ultrasound device of claim 1, wherein the tap detection circuitry is configured to detect the one or more taps using a threshold acceleration between or equal approximately 0-62.5 mg.

8. The ultrasound device of claim 1, wherein the tap detection circuitry is configured to detect the one or more taps using a threshold acceleration between or equal approximately 62.5-125 mg.

9. The ultrasound device of claim 1, wherein the tap detection circuitry is configured to detect the one or more taps using a threshold acceleration between or equal approximately 125-187.5 mg.

10. A processing device in operative communication with an ultrasound device, the processing device configured to:
receive, from the ultrasound device, an indication of detection of one or more taps on an exterior of the ultrasound device during ultrasound imaging; and
perform an action controlling an aspect of the ultrasound imaging based on receiving the indication of the detection of the one or more taps from the ultrasound device.

11. The processing device of claim 10, wherein the processing device is configured, when performing the action controlling the aspect of the ultrasound imaging, to:
control recording of a cine, freeze a current ultrasound image on a screen of the processing device, save to memory an ultrasound image that is frozen on the screen of the processing device, modify an imaging depth, modify a gain, toggle color Doppler imaging on or off, switch imaging modes, and/or switch imaging presets.

12. The processing device of claim 10, wherein the processing device is configured, when performing the action, to modify the processing device's own configuration.

13. The processing device of claim 10, wherein the processing device is configured, when performing the action, to modify the ultrasound device's configuration.

14. The processing device of claim 10, wherein the processing device is configured, when performing the action, to modify both the processing device's own configuration and the ultrasound device's configuration.

15. The processing device of claim 10, wherein the processing device is further configured to provide a user with options for the action that the processing device will be configured to perform based on receiving the indication of the detection of the one or more taps on the exterior of the ultrasound device.

16. The processing device of claim 10, wherein the processing device is further configured to provide a user with options for a threshold tap that the ultrasound device will use to detect one or more taps.

17. The processing device of claim 10, wherein the processing device is further configured to provide a user with options for timing that the ultrasound device will use to detect double taps.

18. The processing device of claim 10, wherein the processing device is further configured to provide a user with options for a duration that the processing device will be configured to perform the action based on receiving the indication of the detection of the one or more taps on the exterior of the ultrasound device.

19. The processing device of claim 10, wherein the processing device is configured, when receiving the indication of the detection of the one or more taps during the ultrasound imaging, to receive the indication of the detection of the one or more taps after a session of the ultrasound imaging has begun.

20. The processing device of claim 10, wherein the processing device is configured, when receiving the indication of the detection of the one or more taps during the ultrasound imaging, to receive the indication of the detection of the one or more taps after the ultrasound device has begun to collect ultrasound data.

21. The processing device of claim 10, wherein the processing device is configured, when receiving the indication of the detection of the one or more taps during the ultrasound imaging, to receive the indication of the detection of the one or more taps while the ultrasound device is placed on a subject for the ultrasound imaging.

22. The processing device of claim 10, wherein the action does not comprise initiating the ultrasound imaging.
